(19) United States
(12) Patent Application Publication
(10) Pub. No.: US 2024/0003899 A1
(43) Pub. Date: Jan. 4, 2024

(54) METHOD FOR DETECTION OF ANALYTES IN A SINGLE TISSUE SAMPLE FROM ITO SLIDES USING MSI-LCM

(71) Applicants: UNIVERSITEIT MAASTRICHT, MAASTRICHT (NL); ACADEMISCH ZIEKENHUIS MAASTRICHT, MAASTRICHT (NL)

(72) Inventors: Berta CILLERO PASTOR, MAASTRICHT (NL); Ronald Martinus Alexander HEEREN, WEESP (NL); Stephanie Theresia Petronella MEZGER, EINDHOVEN (NL)

(21) Appl. No.: 18/036,930

(22) PCT Filed: Oct. 13, 2021
(86) PCT No.: PCT/EP2021/078270

§ 371 Date: May 15, 2023

(30) Foreign Application Priority Data
Oct. 13, 2020 (EP) 20201538.4

(51) Int. Cl. G01N 33/68; G01N 1/28

(52) U.S. Cl. CPC G01N 33/6851; G01N 2560/00; G01N 2001/2886; G01N 1/286

(57) ABSTRACT

The present invention relates to a method for the detection of analytes in a tissue sample, wherein a combination of mass spectrometry imaging (MSI) analysis and laser capture microdissection (LCM) is carried out on a tissue sample on a conductive glass slide, using the same section for MSI and LCM. The method can be used for the detection of proteins, lipids, metabolites and glycans.

The present invention relates to a method for the detection of analytes in a tissue sample using laser capture microdissection (LCM), in particular in combination with mass spectrometry imaging (MSI).

Matrix assisted laser desorption/ionization (MALDI) mass spectrometry imaging (MSI) is a well-recognized technology, and has become a powerful method for tissue-based disease classification and patient stratification. The power of MALDI-MSI resides in the ability to detect proteins, lipids, metabolites and drugs while preserving the information on their spatial localization. By scanning the sample with a well-focused laser beam individual mass spectra are recorded from predefined coordinates providing within a short time, a detailed molecular fingerprint of the tissue investigated. Recent instrumental improvements have resulted in spatial resolution below 10 μm and scan times of just several minutes, making it suitable to significantly lower costs and improve decision making strategies.

Mass spectrometry imaging (MSI) offers unlabelled in-depth molecular detection from tissue sections while maintaining their spatial information. Different sample preparation protocols allow the analysis of a wide range of molecular classes, from small metabolites to large proteins [1]. Although subsequent molecular identification can be done on the same tissue section using tandem mass spectrometry (MS/MS), this direct identification remains limited to the most abundant molecules, especially for intact proteins. Hence, increasing the number of identified molecules often requires separate experiments, with additional dimensions of separation such as liquid chromatography (LC) using for example, tissue homogenates. The major drawback of this approach however is the loss of spatial information.

In this regard, the development of laser capture microdissection (LCM) enables the selection of specific regions and allows subsequent molecular identification strategies [2]. A first study by Banks et al. investigated its feasibility for protein-based analysis and showed no effect of the infra-red laser on the protein integrity [3]. Alternatively, UV lasers that cut around regions of interest (ROI) from tissues placed on membrane glass slides, can also be used. The tissue is subsequently collected by gravity or laser/pressure catapulting. These systems were used in more recent studies coupled with MSI, showing the potential of MSI-guided LCM [4]-[7]. Here the spatial molecular information obtained from MSI is used for ROI selection, acquiring more in-depth molecular information and improving the overall molecular identification. In addition, the necessity of a proper co-registration between histology, MSI data and LCM was described [6]. Especially now the MSI field is moving to higher spatial resolution and single-cell imaging, using a consecutive section might introduce issues due to section-to-section variability.

For example, Greco et al. (“Enabling MSI-Guided Laser Capture Microdissection”, 2019 Oct. 1, pages 1-2, DOI: 10.13140/rg.2.2.35079.55200) discloses using consecutive tissue sections where one section is mounted on an ITO coated slide and used for MSI and one section is mounted on a PEN coated slide and used for LCM. Further, L′Imperio et al. (“MALDI-MSI approach to renal biopsies of patients with fabry disease”, NEPHROLOGY DIALYSIS TRANSPLANTATION, vol. 33, no. suppl_1, 2018 May 1, pages i1-i660, DOI: 10.1093/ndt/gfy104) further establish that MSI can be performed on ITO coated glass slides.

Previous MSI analysis coupled to LCM used non-conductive membrane slides often with consecutive sections for MSI and LCM. For example Dilillio et al. (“Mass Spectrometry Imaging, Laser Capture Microdissection, and LC-MS/MS of the Same Tissue Section”, JOURNAL OF PROTEOME RESEARCH, vol. 16, no. 8, 2017 Jul. 5, pages 2993-3001, DOI: 10.1021/acs.jproteome.7b00284) disclose that MSI and LCM can be used on the same tissue section when using PEN coated slides. These membrane slides maintain tissue morphology and thus the best tissue quality for molecular identification. A main disadvantage, however, is that these slides are not compatible with all MS imaging instruments. Most MSI instruments need an electrically conductive surface such us an indium-tin-oxide (ITO) coated glass slide and its implementation in the LCM workflow would be beneficial as it avoids the necessity for additional tissue sections.

The present invention now provides a method for the detection of analytes in a tissue sample, comprising the steps of:
applying the tissue sample to a glass slide having an electrically conductive coating;
carrying out a mass spectrometry imaging (MSI) analysis of the tissue sample on the glass slide;
subjecting the tissue sample to laser capture microdissection (LCM) to dissect sample material from the tissue sample on the same glass slide; and
analysing the dissected sample material to detect the analytes.

Preferably the laser capture microdissection is carried out in ablation mode. When used herein, carrying out LCM in ablation mode refers to ablating the tissue area of interest (also referred herein as region of interest or ROI). Thus when referring to ablating in the method according to the invention, what meant is, using a laser to target the glass slide in contact with the tissue, particularly the part of the glass slide in contact with the ROI, and all the area of interest is bombarded. This is different from a cut out method, where only the border of the ROI is fired and the ROI remains intact but is captured.

The present invention thus provides a new and effective MSI-LCM workflow using a non-membrane slide. In the method, the analytes are ablated with LCM directly from a conductive slide in combination with MSI on the same slide.

The invention is particularly suitable for detecting analytes in a biological tissue sample. The tissue sample is a biological tissue sample such as animal or a human. Tissue (e.g. muscle, tendon, etc.) and organs (e.g. liver, kidney, brain, pancreas, skin, heart, etc.) can be used as a sample. The tissue or organ sample can be obtained by methods known by a person skilled in the art. It is generally a sectioned tissue slice with a thickness of several μms. The tissue may have undergone a histology staining step.

The tissue can for instance be frozen tissue or formalin fixed paraffin embedded (FFPE) tissue. In case of FFPE the method may include a step of removing the paraffin by using an appropriate solvent such as xylene and/or isopropanol, optionally at elevated temperature, e.g. 60° C.

The analytes to be detected can be proteins, lipids, glycans or metabolites. The method is particularly suitable to detect proteins.

The proteins to be detected originate from (sub-)cellular components (mitochondria, cytoplasm, nuclei or cytoskeleton) or they can be extracellular matrix proteins.

The method of the invention uses a glass slide with an electrically conductive coating. Such slides are known for use with MSI. Preferably, the glass slide is coated with an indium tin oxide coating.

The MSI analysis can be selected from known mass spectrometry methods such as MALDI (Matrix-Assisted Laser Desorption-Ionization), LDI (Laser Desorption-Ionization), LESA (Liquid Extraction Surface Analysis), LAESI (Laser Ablation Electrospray Ionization), DESI (Electrospray Desorption-Ionization), NanoDESI and SIMS (Secondary Ion Mass Spectrometry). Preferably, the method of the invention uses a MALDI-MSI or SIMS-MSI method.

In case of MALDI MSI, the method further comprises a step of applying a matrix onto the tissue sample before carrying out the MALDI-MSI analysis and a step of removing the matrix after carrying out the MALDI-MSI analysis.

Matrix materials for MALDI are known in the art. The matrix materials facilitate the production of intact gas-phase ions from the material in the sample to be analysed. A laser beam serves as the desorption and ionization source. The preferred matrix material is thus capable of absorbing radiation at a specific wavelength from the laser source (typically ultraviolet or infrared laser source). Depending on the method used to apply the matrix, a further requirement may be that it is soluble in appropriate solvents and that it is stable in vacuum.

Examples of matrix materials are: α-cyano-4-hydroxycinnamic acid (CHCA), sinapic acid (4-hydroxy-3,5-dimethoxycinnamic acid), 2,5-dihydroxybenzoic acid (DHB), 2-(4-hydroxy phenyl azo) benzoic acid (HABA), succinic acid, 2,6-dihydroxy acetophenone, ferulic acid, caffeic acid (3,4-dihydroxy-cinnamic acid), 2,4,6-trihydroxy acetophenone, 3-hydroxypicolinic acid, 2-aminobenzoic acid, nicotinic acid, trans-3-indoleacrylic acid, isovanillin, dithranol, 9-aminoacridine (9-AA) and β-carboline (Norharmane).

Preferred matrix materials are 9-AA, Norharmane and sinapic acid.

In the method of the invention, the matrix is preferably removed prior to the LCM step. This can be done with a suitable solvent, such as ethanol.

As described above, the present invention includes a step of subjecting the tissue sample to laser capture microdissection (LCM) to dissect sample material from the tissue sample. Laser capture microdissection is a known method and is for instance described in EP1288645.

It is preferable to adjust the conditions of the LCM such that as little damage as possible occurs to the analytes in the tissue sample. Settings differ from those used with membrane slides in known LCM methods.

Preferably, the laser capture microdissection is carried out under the using “draw and scan” conditions, also called laser ablation or dot scan dissection. When used herein, carrying out the LCM in ablation mode refers to ablating the region of interests. Using ablation the region of interest is collected for further analysis. It was surprisingly found by the inventors that the protein integrity is preserved using this method. An advantage of this method is that it uses conventional conducive slides such as ITO glass slides which can be used in any type of mass spectrometer, so it does not rely on PEN coated slides which are not conducive and can only be used in certain types of mass spectrometers. The method thus can be applied more universally.

The described method is preferably used such that a region of interest (ROI) is defined using MSI which is then subjected to further molecular analysis by e.g. MS-MS. By performing both on the same tissue section, the accuracy is greatly improved compared to using consecutive tissue sections. Therefore in an embodiment the MSI analysis is used to define a region of interest (ROI). Preferably the ROI is ablated in the LCM step.

In an embodiment the ablated tissue sample is collected. The collected tissue sample may for example be treated for storage such as cryopreserving, or may be treated for further analysis e.g. by MS-MS to identify and quantify molecules of interest in the collected tissue sample.

25 Typical conditions are a wavelength 349 nm, power 50, aperture 38, speed 17, specimen balance 0, line spacing 5, head current 60%, and pulse frequency 310 Hz. Power, speed and frequency are likely the most important conditions.

The dissected sample material after LCM is subjected to known methods to detect the analytes. These methods include known proteomics, metabolomics, glycomics and lipidomics and liquid chromatography “omics” analysis. Such methods are known to the skilled person.

### DRAWINGS

FIG. 1 shows the workflow of the method of the invention.

FIG. 2 shows proteins identified from PEN membrane (comparative) and ITO slides for A) frozen tissue and B) FFPE tissue. Data are presented as mean±SD. * indicates p<0.05 using t-test.

FIG. 3 shows the comparison of two laser settings for ablation from an ITO slide as the number of proteins from A) frozen tissue and B) FFPE tissue. Data are presented as mean±SD, * indicates p<0.05 the using t-test.

FIG. 4 shows the number of proteins identified after MSI from ITO and IntelliSlides™ (INT) followed by LCM. Lipid MSI on frozen tissue in A) positive ion mode, B) negative ion mode, and C) metabolite MSI on FFPE tissue in negative ion mode. Data are presented as mean±SD. * indicates p<0.05 when comparing results before versus after MSI.

FIG. 5 shows segmentation data from positive ion mode lipid from ITO slides (A). The numbers indicate clusters 1 (purple) and 2 (green). B) The number of proteins identified from clusters 1 and 2, data is presented as mean±SD.

FIG. 6 shows segmentation analysis of sham and I/R hearts divided the tissue over 7 clusters, separating infarct, unaffected tissue, blood and matrix. A) The segmentation results showed representation of the infarct core by the green and infarct border by orange clusters, while the red, yellow and turquoise clusters correspond to unaffected tissue. The blood and matrix were represented by the purple and blue clusters, respectively. B) Based on the segmentation results (black lines) different ROIs were selected for proteomics analysis. The colored regions (approximately 0.5 mm2) were ablated using LCM.

FIG. 7 shows Proteins identified in the different clusters, with (A) the number of proteins identified, and (B) heatmap showing the abundance ratio (log 2) for classically known cardiac biomarkers, * indicates adjusted p-value 0.05.

FIG. 8 shows categorized representation of the cellular components found after LMD on both frozen (A) and FFPE (B) tissue. For this analysis, all significant components (p<0.05) were taken into account.

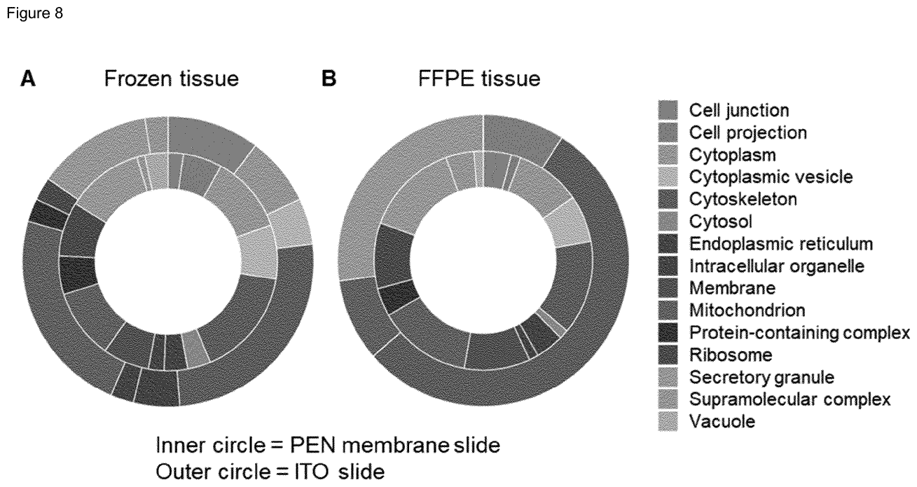

FIG. 9 shows cellular components found in frozen tissue before (A) and after negative lipid MSI (B). All components with a p-value <0.05 were taken into account.

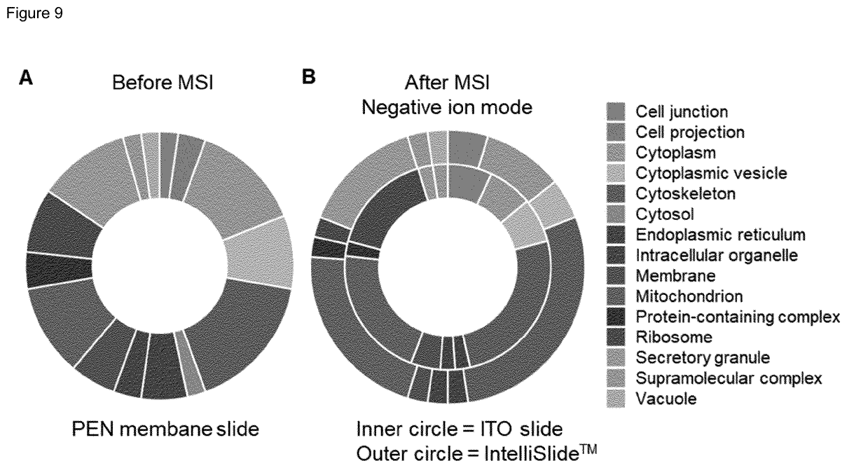

FIG. 10 shows all cellular components found in FFPE tissue before (A) and after (B) metabolite MSI. Components with p-value<0.05 were taken into account.

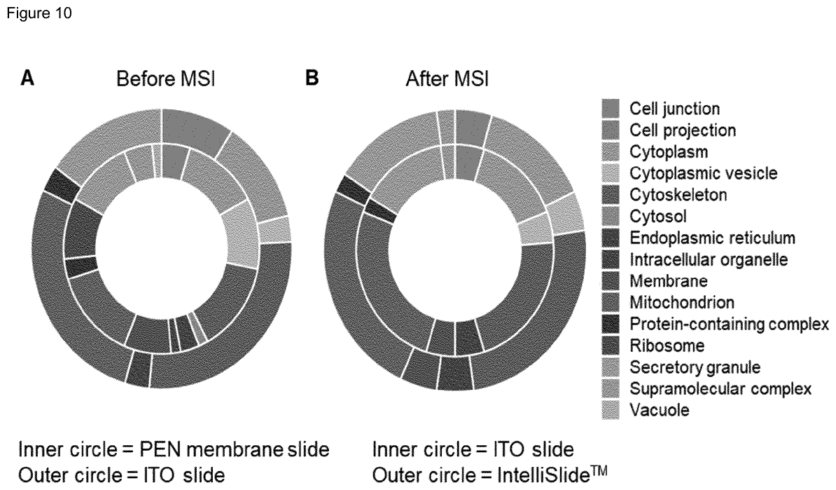

### EXAMPLES

### Example 1

### Materials

### Chemicals and Solvents

All solvents (ULC grade) were purchased from Biosolve unless stated otherwise. 9-aminoacridine (9AA), ammonium bicarbonate (ABC), α-cyano-4-hydroxycinnamic acid (CHCA), citric acid, dithiothreitol (DTT), Eosin-Y (Avantor), formic acid (FA, ULC grade), iodoacetamide (IAM), norharmane, trifluoroacetic acid (TFA, ULC grade), and xylene were purchased from Sigma-Aldrich. RapiGest™ SF was purchased from Waters. Trypsin (modified porcine, Sequencing Grade) was purchased from Promega. Polyethylene naphthalate (PEN) microdissection membrane slides and 0.2-mL tubes were purchased from Leica Microsystems. Indium tin oxide (ITO) glass slides were obtained from Delta Technologies (Loveland, USA) and IntelliSlides™ from Bruker Daltonics GmbH (Bremen, Germany).

### Tissue Samples

All animal experiments were approved by the Institutional Animal Care and User Committee of Maastricht University, and they were performed adhering to the Dutch law. Residual Wistar Han rat cardiac tissue was provided by the group of General Surgery, Maastricht University Medical Center, Maastricht, The Netherlands. Rat cardiac tissue was flash frozen after organ removal. Using a cryotome (Leica Microsystems, Wetzlar, Germany) 10 μm thick sections were cut at −20° C. and thaw mounted onto either PEN membrane, ITO slide or IntelliSlide™. The slides were stored at −80° C. until further analysis.

Residual mouse cardiac tissue was provided by the department of Physiology, Maastricht University, Maastricht, The Netherlands. After removal, the tissue was fixed in 4% paraformaldehyde for forty-eight hours, embedded in paraffin and stored at room temperature until sectioning. From this formalin fixed paraffin embedded (FFPE) tissue, sections of 4 μm thick were cut with a rotary microtome (Microm GMBH HM 355) and placed on either PEN membrane, ITO slide or IntelliSlide™. The slides were stored at +4° C. until further analysis.

### Analysis

### Lipid Mass Spectrometry Imaging on Frozen Tissue

Frozen rat cardiac tissue deposited on an ITO slide or IntelliSlide™ was covered with 15 layers of 7 mg/mL norharmane in 2:1 chloroform:methanol using a Suncollect pneumatic sprayer (SunChrom GmbH, Germany). The sections were imaged at 75 μm raster size on a RapifleX tissueTyper (Bruker Daltonics GmbH, Bremen, Germany) in positive or negative ion reflector mode at a m/z range of 400-2000, summing 500 laser shots per position. The instrument was calibrated using red phosphorus. After MSI the slides were stored at −80° C. until LCM.

### Metabolite Mass Spectrometry Imaging on FFPE Tissue

The FFPE mouse cardiac tissue underwent deparaffinization with two 8 min Xylene washes, as described previously [8], followed by the application of 11 layers of 10 mg/mL 9-AA in 70% methanol using a Suncollect pneumatic sprayer (SunChrom GmbH, Germany). All sections were imaged at 75 μm raster size on a RapifleX tissueTyper (Bruker Daltonics GmbH, Bremen, Germany) in negative ion reflector mode at a m/z range of 40-1000, summing 500 laser shots per position. Instrument calibration was done using red phosphorus. After MSI the slides were stored at +4° C. until LCM.

### Laser Capture Microdissection

LCM was performed using the Leica LCM 7000 (Leica Microsystems, Wetzlar, Germany). For FFPE tissues, the paraffin was removed by 2 h of heating at 60° C. followed by two 5 min washes with xylene and two 2 min washes with isopropanol [7]. Before LCM the tissue sections were dried in a desiccator.

A total of 0.1, 0.2, 0.5, or 1.0 mm² dissected material was collected in triplicate, from FFPE and frozen material, before and after hematoxylin and eosin (H&E) staining.

The areas were dissected using the following laser settings: wavelength 349 nm, power 40, aperture 30, speed 5, specimen balance 0, line spacing 5, head current 60%, and pulse frequency 501 Hz (later referred to as settings A).

A second set of laser parameters was also used for ITO and IntelliSlides™: wavelength 349 nm, power 50, aperture 38, speed 17, specimen balance 0, line spacing 5, head current 60%, and pulse frequency 310 Hz (referred to as settings B).

For PEN membrane slides ‘draw and cut’ was used and ‘draw and scan’ was used for ITO slides and IntelliSlides™.

Dissected areas were collected in the caps of 0.2-mL centrifuge tubes, prefilled with 20 μL buffer (50 mM ABC for frozen, 50 mM citric acid for FFPE) and stored at −20° C. until further processing for LC-MS/MS.

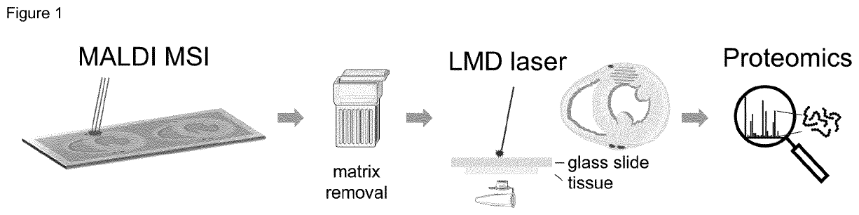

LCM after MALDI MSI was performed on ITO slides and IntelliSlides™ after matrix removal with 70% ethanol, as shown in FIG. 1. A region of interest (ROI) was selected based on segmentation data and co-registered with the LCM using an in-house build MATLAB script [6]. Areas of 0.5 mm² were ablated from the ITO slide using laser settings B, as described above, collected in 20 μL 50 mM ABC buffer and stored at −20° C. until further processing for LC-MS/MS.

### Sample Processing for Proteomics

The dissected material was further processed based on the protocol as previously described by Longuespee et al [7]. In short, for FFPE samples antigen retrieval was performed by heating to 99° C. for an hour while shaking at 800 rpm in a Thermoshaker (Eppendorf, Hamburg, Germany). For both FFPE and frozen samples, RapiGest™ (final concentration 0.01%) was added and incubated for 10 min at room temperature (RT=21° C.), the pH of FFPE samples was adjusted by addition of ABC. All samples were reduced using DTT (10 mM) at 56° C. for 40 min at 800 rpm and alkylated using IAM (20 mM) at RT for 30 min at 800 rpm. DTT (10 mM) was used to quench the excess of IAM at RT for 10 min at 800 rpm. Digestion using trypsin (15 μg/ml) was performed overnight at 37° C. and 800 rpm. The second digestion step (trypsin 5 μg/ml) was performed in 80% ACN for 3 hours at 37° C. and 800 rpm. With the addition of TFA (final concentration 0.5%) the digestion was stopped in 45 min at 37° C. and 800 rpm. After centrifugation (15000×g, 10 min at 4° C., Thermo scientific Heraeus Biofuge stratos) the supernatant was collected and concentrated to a final volume of approximately 30 μL using a speedvac (Hetovac VR-1 Hetosicc). The concentrated samples were stored at −20° C. until LC-MS/MS analysis.

### LC-MS/MS Analysis

Peptide separation was performed on a Thermo Scientific (Dionex) Ultimate 3000 Rapid Separation UHPLC system equipped with a PepSep C18 analytical column (15 cm, ID 75 μm, 1.9 μm Reprosil, 120 Å). An aliquot of 10 μL of sample was desalted using an online installed C18 trapping column, the peptides were separated on the analytical column with a 90 min linear gradient from 5% to 35% ACN with 0.1% FA at 300 nL/min flow rate.

The UHPLC system was coupled to a Q Exactive™ HF mass spectrometer (Thermo Scientific). Mass spectra were acquired in positive ionization mode, full MS scan between m/z 250-1250 at resolution of 120.000 followed by MS/MS scans of the top 15 most intense ions at a resolution of 15.000 to obtain DDA results.

### Data Analysis

The triplicates were analyzed individually and protein identification was done using Proteome Discoverer 2.2 (Thermo Scientific). The search engine Sequest was used with the SwissProt Rattus norvegicus (SwissProt TaxID=10116) or Mus musculus (SwissProt TaxID=10090) database. The following settings were used for the database search: Trypsin was used as enzyme with a maximum of 2 missed cleavages and a minimal peptide length of 6 amino acids. Mass tolerance for precursor of 10 ppm, for fragment of 0.02 Da. Dynamic modifications of methionine oxidation and protein N-terminus acetylation, static modifications of cysteine carbamidomethylation.

Results of the numbers of proteins identified per triplicates are presented as mean±standard deviation (SD). Comparisons were performed with the t-test or one-way ANOVA and p<0.05 was considered statistically significant. All statistical analyses were performed using GraphPad Prism (version 5.00; GraphPad Software, Inc., San Diego, CA).

Proteins commonly identified in the triplicates were used for gene ontology cellular component analysis. UniProt ID mapping was used to obtain the gene names which were then submitted to EnrichR [9] where cellular components with p-value <0.05 were considered for further analysis. The components were categorized based on a higher level in the Gene Ontology Cellular Component tree for a more concise and structured analysis. Pathway analysis was performed for the differentiation of the clusters after MSI. EnrichR used Reactome's cell signaling database and pathways with p-value <0.05 were used for the analysis.

MSI data were analyzed using SCiLS lab MVS, version 2020a (Bremen, Germany) after TIC normalization. Segmentation by bisecting k-means with correlation distance was performed to obtain ROI information. mMass10 was used to generate a peak list (15 precision baseline correction with 25 relative offset, Savitzky-Golay smoothing with a window size of 0.2 m/z and 2 cycles, at last peaks were picked with a S/N threshold of 3.5, relative intensity threshold of 0.5% and picking height 75).

### Results

Protein Identification from PEN Membrane and ITO Slides

The invention was evaluated and compared to conventionally used PEN membrane slides for cardiac tissue. The number of identified proteins was determined for different amounts of tissue dissected (0.1, 0.2, 0.5 and 1.0 mm²) for both frozen and FFPE tissue.

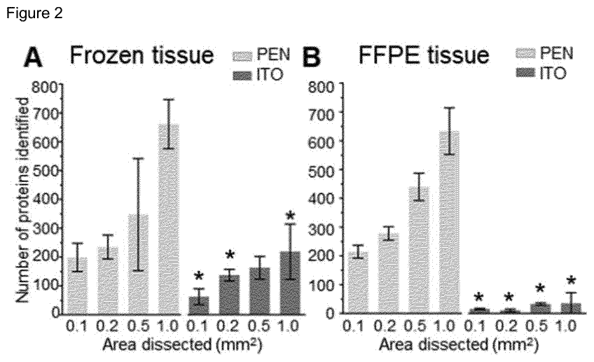

FIG. 2 shows the feasibility of protein identification from ITO slides. The number of proteins identified was found to increase as dissected area size increased. This trend was significant for frozen tissue from both slide types (p=0.0035 for PEN membrane and p=0.0452 for ITO slides), and for FFPE tissue only from PEN membrane slides, p<0.0001. As expected, the number of identified proteins was significantly higher in PEN membrane slides for both frozen and FFPE tissue (p<0.05) for all areas (*). Interestingly, the number of identified proteins for frozen and FFPE tissue were similar in PEN membrane slides, while the number of identified proteins from ITO slides was lower for FFPE compared to frozen tissue.

An enrichment analysis on commonly found proteins within the triplicates was performed to assess the cellular component origin and to verify whether the protein integrity was maintained after tissue dissection. Table 1 depicts the top 10 most significant cellular components and shows the preservation of cellular components from mitochondrial and secretory granule proteins for all studied samples. Other less abundant cellular components were different between the PEN membrane and ITO slides.

TABLE 1

| | |
|---|---|
| Frozen tissue | FFPE tissue |

| | PEN | | PEN | |
|---|---|---|---|---|
| | membrane | ITO | membrane | ITO |
| Cell junction | x | x | x | |
| Cell projection | | | | x |
| Cytoplasm | | x | | |
| Cytoplasmic vesicle | x | | | |
| Cytoskeleton | | x | | x |
| Cytosol | x | | x | |
| Mitochondrion | x | x | x | x |
| Ribosome | x | | x | |
| Secretory granule | x | x | x | x |

A more detailed analysis of all significant cellular components revealed that for frozen tissue there were 87 and 38 cellular components found from PEN membrane and ITO slides, respectively. In comparison, analysis of FFPE tissue resulted in 72 and 11 cellular components, respectively. Despite this variability, the categorized analysis again showed a good preservation between the samples with a large contribution from cytoskeletal, mitochondrial, and secretory granule proteins.

### Effect of the LCM Laser on Protein Identification

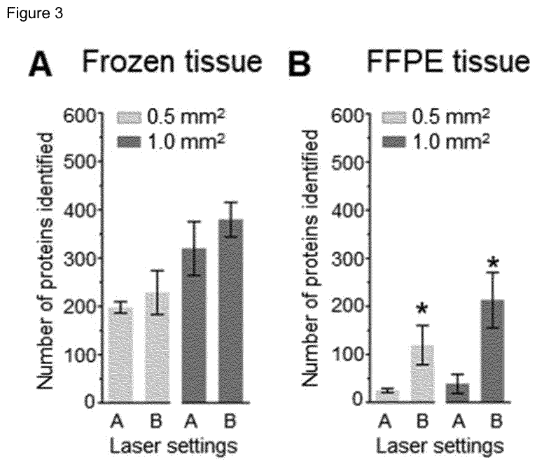

Next, the laser parameters were adjusted with the aim to improve the number of proteins identified, which was based on visual inspection of the residue left on the ITO slide after collection with LCM. The results as shown in FIG. 3 indicate an improvement in number of identified proteins when using settings B, for frozen (p>0.05) and especially for FFPE tissue (p=0.0161 and p=0.0077 for 0.5 and 1.0 mm2, respectively). Cellular component analysis showed no differences for the top 10 most significant components (Table 2).

Based on the improvement seen for FFPE tis-sue on ITO slide settings B were used without further optimization.

TABLE 2

| | |
|---|---|
| Frozen tissue | FFPE tissue |

| Laser setting | A | B | A | B |
|---|---|---|---|---|
| Cell junction | x | x | | |
| Cytoplasm | x | x | x | x |
| Cytoplasmic vesicle | x | x | | |
| Cytoskeleton | | | x | x |
| Mitochondrion | x | x | x | x |
| Protein-containing complex | | | | x |
| Secretory granule | x | x | | x |

Proteomics after MALDI-MSI

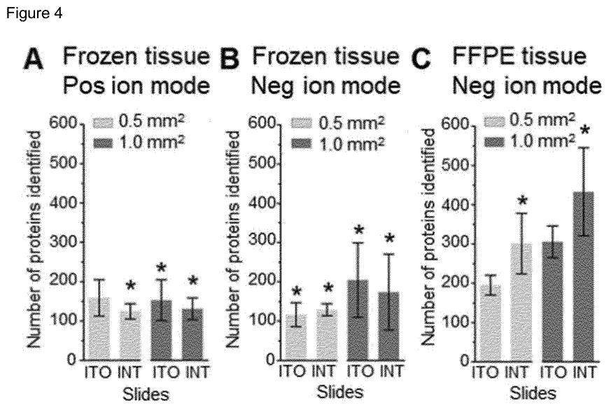

FIG. 4 shows that proteins can still be identified from tissue sections that were previously used for lipid or metabolite MSI. After MSI, both slide types showed a comparable number of identified proteins for frozen tissue and FFPE tissues on IntelliSlides™ (marked as INT in the figure). In contrast to previous results, no increase was seen for frozen tissue when bigger areas were dissected. Moreover, comparing the number of identified proteins from frozen tissue before (FIG. 3A, laser settings B) and after MSI showed a significant decrease in the number of identified proteins, as indicated with an asterisk (*) in FIGS. 4A and B. Despite this reduction, the number of identified proteins remained above 100.

On the other hand, FFPE tissue after metabolite MSI (FIG. 4C) showed an increase in the number of proteins when more tissue was dissected (p=0.0159 for ITO slides, p>0.05 for IntelliSlides™), unexpectedly, a higher number of identifications was shown after metabolite MSI.

After MALDI MSI, the top 10 most significant cellular components were found to be preserved compared to those from tissue before MALDI MSI as shown in Table 3.

TABLE 3

| | Frozen tissue | | | FFPE Tissue | | |
|---|---|---|---|---|---|---|
| | | After | | | After | |
| | Before | ITO | IntelliSlide | Before | ITO | IntelliSlide |
| polarity | ITO | +/− | +/− | ITO | − | − |
| Cell junction | x | | | | x | x |
| Cytoplasm | x | x/x | x/x | x | | |
| Cytoplasmic | x | 0/x | | | | |
| vesicle | | | | | | |
| Cytoskeleton | | x/x | x/x | x | x | x |
| Mitochondrion | x | x/x | x/x | x | x | x |
| Protein- | | | | x | | |
| containing | | | | | | |
| complex | | | | | | |
| Secretory | x | 0/x | 0/x | x | | |
| granule | | | | | | |

For frozen tissue, cytoplasmic and mitochondrial proteins were preserved. Interestingly, more cytoskeletal proteins and less secretory granule proteins were identified after lipid MSI compared to before MSI. For FFPE tissue, cytoskeletal and mitochondrial proteins were preserved, while more cell junctional proteins were found and less cytoplasmic and secretory granule proteins.

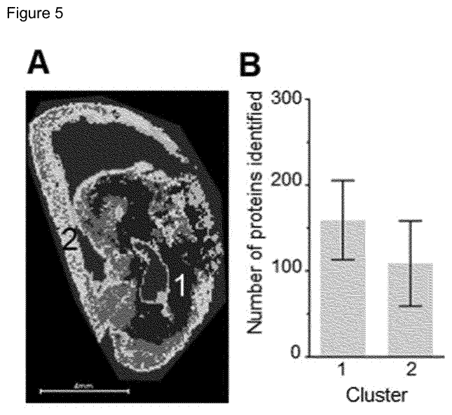

Finally, segmentation data from positive ion mode lipid MALDI MSI was used for the selection of two clusters, as indicated in FIG. 5A, to illustrate the use of conductive slides for MSI-guided LCM. FIG. 5B shows the number of proteins identified in both clusters. Further analysis of some biological differences between the clusters was done with a pathway analysis, the detailed results can be found in Table 4. In total, 103 pathways were identified, of which 29 and 33 pathways were specific for cluster 1 and 2, respectively.

TABLE 4
Pathway analysis from clusters 1 and 2 dissected from ITO slides
after positive lipid MSI. All significant pathways (p <
0.05) were included and displayed in an alphabetical order.

| Common pathways, n = 41 | Specific for cluster 1, n = 29 | Specific for cluster 2, n = 33 |
|---|---|---|
| AUF1 (hnRNP D 0) binds and | Activation of caspases | Assembly Of The |
| destabilizes mRNA | through apoptosome- | HIV Virion |
| | mediated cleavage | |
| Beta oxidation of lauroyl- | Acyl chain remodeling of CL | Association of licensing |
| CoA to decanoyl-CoA-CoA | | factors with the pre- |
| | | replicative complex |
| Binding and Uptake of | Amino acid synthesis and | Cell-extracellular matrix |
| Ligands by Scavenger | interconversion | interactions |
| Receptors | (transamination) | |
| CHL1 interactions | Apoptotic factor-mediated | Constitutive Signaling by |
| | response | NOTCH1 HD Domain |
| | | Mutants |
| Citric acid cycle (TCA cycle) | Beta oxidation of decanoyl- | Diseases of carbohydrate |
| | CoA to octanoyl-CoA-CoA | metabolism |
| Creatine metabolism | Beta oxidation of hexanoyl- | Disorders of transmembrane |
| | CoA to butanoyl-CoA | transporters |
| Downregulation of ERBB4 | Beta oxidation of octanoyl- | Downregulation of |
| signaling | CoA to hexanoyl-CoA | ERBB2:ERBB3 signaling |
| Erythrocytes take up carbon | Branched-chain amino acid | Glycogen breakdown |
| dioxide and release oxygen | catabolism | (glycogenolysis) |
| Erythrocytes take up oxygen | Cellular responses to stress | Glycogen storage diseases |
| and release carbon dioxide | | |
| Gluconeogenesis | Complex I biogenesis | Glycogen synthesis |
| Glucose metabolism | Cytochrome c-mediated | Hemostasis |
| | apoptotic response | |
| Glycolysis | Detoxification of Reactive | IRAK2 mediated activation of |
| | Oxygen Species | TAK1 complex |
| Lipid digestion, mobilization, | Fatty acid, triacylglycerol, | IRAK2 mediated activation of |
| and transport | and ketone body metabolism | TAK1 complex upon TLR7/8 |
| | | or 9 stimulation |
| Metabolism | Fructose metabolism | MAP3K8 (TPL2)-dependent |
| | | MAPK1/3 activation |
| Metabolism of amino acids | Glyoxylate metabolism and | Membrane binding and |
| and derivatives | glycine degradation | targetting of GAG proteins |
| Metabolism of carbohydrates | Histidine, lysine, | Membrane Trafficking |
| | phenylalanine, tyrosine, | |
| | proline and tryptophan | |
| | catabolism | |
| Metabolism of polyamines | Hormone-sensitive lipase | Myoclonic epilepsy of Lafora |
| | (HSL)-mediated | |
| | triacylglycerol hydrolysis | |
| mitochondrial fatty acid beta- | Ion homeostasis | NF-kB is activated and |
| oxidation of saturated fatty | | signals survival |
| acids | | |
| mitochondrial fatty acid beta- | Ketone body metabolism | NRIF signals cell death from |
| oxidation of unsaturated fatty | | the nucleus |
| acids | | |
| Mitochondrial protein import | Lysine catabolism | p75NTR recruits signalling |
| | | complexes |
| Mitophagy | Metabolism of lipids and | p75NTR signals via NF-kB |
| | lipoproteins | |
| Muscle contraction | Methionine salvage pathway | Receptor-ligand binding |
| | | initiates the second |
| | | proteolytic cleavage of Notch |
| | | receptor |
| O2/CO2 exchange in | Mitochondrial Fatty Acid | Recycling of bile acids and |
| erythrocytes | Beta-Oxidation | salts |
| Pink/Parkin Mediated | Neurotransmitter Clearance | Reduction of cytosolic Ca++ |
| Mitophagy | In The Synaptic Cleft | levels |
| Platelet activation, signaling | Pregnenolone biosynthesis | Regulation of innate immune |
| and aggregation | | responses to cytosolic DNA |
| Platelet degranulation | Regulation of pyruvate | Regulation of PLK1 Activity |
| | dehydrogenase (PDH) | at G2/M Transition |
| | complex | |
| PTK6 Regulates RTKs and | Signaling by Retinoic Acid | Signaling by NOTCH1 HD |
| Their Effectors AKT1 and | | Domain Mutants in Cancer |
| DOK1 | | |
| Pyruvate metabolism | Synthesis of Ketone Bodies | Spry regulation of FGF |
| | | signaling |
| Pyruvate metabolism and | Transcriptional Regulation | Synthesis And Processing Of |
| Citric Acid (TCA) cycle | by TP53 | GAG, GAGPOL Polyproteins |
| Regulation of cytoskeletal | | TGF-beta receptor signaling |
| remodeling and cell | | in EMT (epithelial to |
| spreading by IPP complex | | mesenchymal transition) |
| components | | |
| Regulation of mRNA stability | | TRAF6 mediated induction of |
| by proteins that bind AU-rich | | TAK1 complex |
| elements | | |
| Respiratory electron | | Translesion synthesis by |
| transport | | REV1 |
| Respiratory electron | | Transport of organic anions |
| transport, ATP synthesis by | | |
| chemiosmotic coupling, and | | |
| heat production by | | |
| uncoupling proteins. | | |
| Response to elevated | | |
| platelet cytosolic Ca2+ | | |
| Scavenging of heme from | | |
| plasma | | |
| SLC transporter disorders | | |
| Striated Muscle Contraction | | |
| The citric acid (TCA) cycle | | |
| and respiratory electron | | |
| transport | | |
| TP53 Regulates Metabolic | | |
| Genes | | |
| Translocation of GLUT4 to | | |
| the plasma membrane | | |
| Vesicle-mediated transport | | |

### Example 2

Myocardial infarction (MI) is the most common cause of cardiovascular deaths and is a result of the blockage of coronary arteries leading to a reduced blood flow to the underlying cardiac tissue. Although early restoration of the blood flow is essential, by thrombolytic therapy or invasive procedures, this sudden reperfusion can cause additional myocardial injury, the so-called ischemia-reperfusion (I/R) injury. After an ischemic event the heart can be classified in infarct (core), peri-infarct (or border) and remote myocardial regions, where complex processes take place, including structural changes and pathological processes, like oxidative stress, activation of cell death, inflammation, and eventually remodeling.

In the present study, the spatialOMx approach was applied after protein MALDI-MSI for the in-depth assessment of pathophysiological protein alterations in cardiac I/R in a rat model. This state-of-the-art approach allowed the identification of changes in protein content and the investigation of pathways involved in I/R injury after an ischemic event, providing insights for the development of strategies to minimize myocardial damage after MI.

Materials and Methods

Chemicals and Solvents

All solvents (ULC grade) were purchased from Biosolve (Valkenswaard, The Netherlands) unless stated otherwise. Ammonium bicarbonate (ABC), 2,6-dihydroxyacethophenone (DHA), dithiothreitol (DTT), Eosin-Y (Avantor), formic acid (FA, ULC grade), Gill's hematoxylin, iodoacetamide (IAM), trifluoroacetic acid (TFA, ULC grade), and xylene were purchased from Sigma-Aldrich (Zwijndrecht, The Netherlands). RapiGest™ SF was purchased from Waters (Milford, USA). Trypsin (Modified porcine, Sequencing Grade) was purchased from Promega (Leiden, The Netherlands). 0.2-mL centrifuge tubes were purchased from Leica Microsystems (Wetzlar, Germany). Indium tin oxide (ITO) glass slides were obtained from Delta Technologies (Loveland, USA).

Protein MALDI Mass Spectrometry Imaging

Tissues were washed 30 sec in 70% ethanol, 30 sec in 100% ethanol, 2 min in Carnoy's solution (being 60% ethanol, 30% chloroform, 10% acetic acid), followed by 30 sec in 100% ethanol, demineralized water, and 100% ethanol. They were afterwards dried in a desiccator. Next, 9 layers of 15 mg/mL DHA in 80% acetonitrile, 0.4% TFA, 0.4% acetic acid were applied using the SunCollect sprayer (SunChrom GmbH, Germany). For co-registration purposes, fiducial markers were placed next to the tissue using water-based Tipp-Ex (BIC, Paris, France). The tissue was analyzed with a RapiFleX tissueTyper (Bruker Daltonics GmbH, Bremen, Germany) in positive ion linear mode, summing 1000 laser shots per position with a laser frequency of 5000 Hz and 80 μm pixel size. Data was acquired in the m/z range from 2000-20000 and protein calibration standard I (Bruker Daltonics) was used for instrument calibration. Slides were stored at −80° C. until LCM analysis.

Haematoxylin and Eosin (H&E) Staining

After MALDI-MSI the matrix was removed with 70% ethanol dips. A standard H&E staining protocol was performed, slides were immersed for 3 min in distilled water, followed by a nuclei stain with 0.1% Gill's haematoxylin in 3 min, bluing is done for 3 min in running tap water and after a rinse with distilled water the cytoplasm was stained in 0.2% eosin for 30 sec, the excess of eosin was removed by short rinse in 70% ethanol, and the sections were dehydrated in 100% ethanol two times 2 min and equilibrated in xylene for two times 5 min. The H&E stained samples were air dried before mounting with cover slip using Entellan. A digital optical image was obtained using the Aperio CS2 slide scanner (Leica Microsystems, Wetzlar, Germany). Annotation of various areas was performed by a pathologist using QuPath v0.2.3.

MSI Data Analysis

All datasets were recalibrated using FlexAnalysis v3.4 (Bruker Daltonics) for optimal spectral comparison. This was done in quadratic correction mode using m/z 5487, 8565, 11307, 12135, 15198 as calibrants with a 500 ppm peak assignment tolerance. After recalibration, the MSI data was analyzed using SCiLS lab MVS version 2021b (Bruker, Bremen, Germany) after total ion current (TIC) normalization with an interval width of 2 Da. The overall average spectrum was imported in mMass to generate a peak list (50 precision baseline correction with 75 relative offset, moving average smoothing window of 5 m/z and 2 cycles, S/N threshold of 2.5, relative intensity threshold of 1.0% and picking height 75). Probabilistic latent semantic analysis (pLSA) with 5 components was performed with random initialization using the overall peaklist on the individual spectra. Discriminative m/z values were evaluated using receiver operating characteristic (ROC) analysis comparing the infarct and unaffected regions from I/R and sham hearts as found by the pLSA taking a random subset of 3000 spectra. The AUC threshold ≥0.8 or <0.2, resulted in m/z values specific for the infarct or the unaffected regions, respectively. Segmentation was performed in SCiLS using bisecting k-means with correlation distance, to obtain region of interest (ROI) information. The coordinates from the ROIs were exported for LCM using an in-house build MATLAB script.

Laser Capture Microdissection

From the selected ROIs, areas of 0.5 mm2 were dissected using the Leica LCM 7000 (Leica Microsystems, Wetzlar, Germany) using the previously established protocol, with the following laser settings: wavelength 349 nm, power 40, aperture 38, speed 5, specimen balance 0, line spacing 5, head current 60%, and pulse frequency 310 Hz in “draw+scan” mode (Mezger et al., 2021). The dissected tissue was collected without prior removal of the DHA matrix in 0.2-ml centrifuge tubes containing 20 μL ethanol, the sample was dried in the speedvac and resuspended in 20 μL 50 mM ABC buffer and stored at −20° C. until further processing.

Sample Processing for Proteomics

The dissected material was further processed as the previously described (Mezger et al., 2021). In short, RapiGest™ was added to enhance enzymatic protein digestion, the samples were reduced using DTT and alkylated using IAM. The excess of IAM was quenched by the addition of DTT. Protein digestion was done using a double trypsin step. The digestion was stopped by the addition of TFA. The supernatant was collected and the concentrated samples were stored at −20° C. until LC-MS/MS analysis.

LC-MS/MS Analysis

An aliquot of 10 μL of the sample was desalted using an online installed C18 trapping column, the peptides were separated with a 90 min linear gradient from 5% to 35% ACN with 0.1% FA at 300 nL/min flow rate. This was performed on a Thermo Scientific (Dionex) Ultimate 3000 Rapid Separation UHPLC system equipped with a PepSep C18 analytical column (15 cm, ID 75 μm, 1.9 μm Reprosil, 120 Å). The UHPLC system was coupled to a Q Exactive™ HF mass spectrometer (Thermo Scientific) with Nanospray Flex source. Mass spectra were acquired in positive ionization mode, full MS scan between 250-1250 m/z at resolution of 120.000 followed by MS/MS scans of the top 15 most intense ions at a resolution of 15.000 in DDA mode.

Proteomics Data Analysis

Protein identification was performed using Proteome Discoverer 2.2 (Thermo Scientific). The search engine Sequest was used with the SwissProt Rattus norvegicus (SwissProt TaxID=10116) database, august 2020. The database search was performed using trypsin as enzyme and a maximum of 2 missed cleavages. The minimal peptide length was set to 6 amino acids, mass tolerance for precursor of 10 ppm and for fragment of 0.02 Da. Methionine oxidation and protein N-terminus acetylation were set as dynamic modifications, carbamidomethylation of cysteine residues as static modification. The false discovery rate was fixed at 1% and used as a measure for certainty of the identification, only proteins with a high protein confidence were used for further analysis. Only protein abundance ratios with a fold-change higher than 1.5 or lower than 0.67 (log 2≥0.58 or ≤−0.58, respectively) and adjusted p-value≤0.05 were considered for further analysis. Protein accession numbers were converted to gene names using UniProt ID mapping. The significantly altered proteins were included in the pathway analysis using EnrichR (Chen et al., 2013; Kuleshov et al., 2016) with the Reactome's cell signaling database. The top 10 up- or downregulated pathways were ranked by the combined score.

Results

Image Guided Proteomics Revealed Up- and Downregulation in the Infarct Area

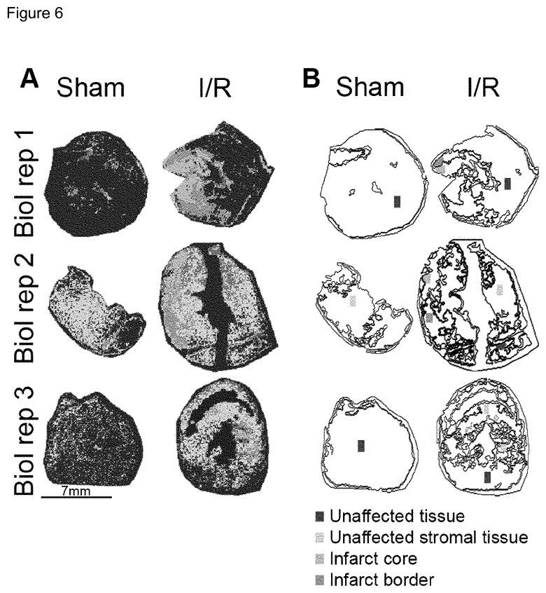

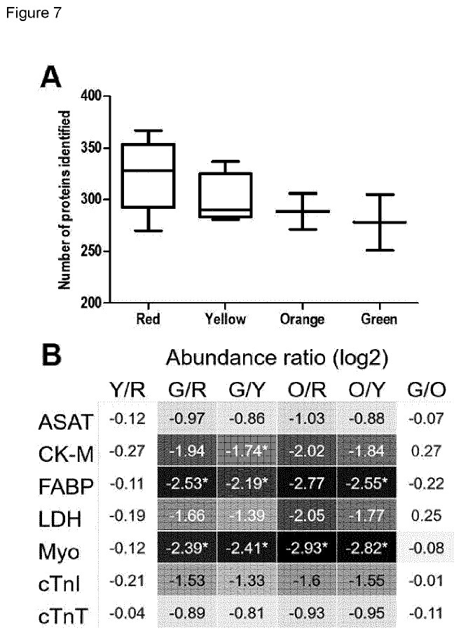

For the first time, we performed protein identification following a spatialOMx approach on the same tissue sections previously used for protein MALDI-MSI. Guided by the segmentation data, regions of 0.5 mm2 were ablated from ITO slides that represent infarct or unaffected tissue (FIG. 6B). Proteomics analysis identified a total of 465 proteins directly from the ITO slides, these proteins were used for subsequent comparative analysis. Between the clusters, the number of identified proteins showed no significant differences (ANOVA p-value=0.34, FIG. 7A).

First, abundance changes in (previously) used cardiac biomarkers that clinically serve for diagnosis and monitoring of myocardial infarction such as cardiac troponins (cTnI and cTnT) were evaluated. The abundance ratios of these proteins are shown in the heatmap in FIG. 7B. A downregulation was observed for all these proteins in the infarct region, with myoglobin (MYO), creatine kinase-M type (CK-M) and fatty acid binding protein (FABP) being statistically significant.

From the identified proteins, 99 were found to be differentially abundant (log 2 ratio ≤−0.58 or ≥0.58 with an adj. p-value ≤0.05) in one of the comparisons as shown in FIG. 6. It should be noted that 16 proteins were not detected in all regions, resulting in a minimal or maximal abundance ratio.

A comparison of the unaffected tissue (red) with the infarct core (green) showed a lower abundance for 17 proteins, amongst others the cardiac biomarkers MYO and FABP. Likewise, 56 proteins were upregulated, like c-reactive protein an important diagnostic marker for inflammation, and indicators for cell damage clusterin and protein S100. The data in Table 5 illustrates that comparisons of the infarct regions with the unaffected tissue results in similar patterns for protein abundances. Between the two clusters within the unaffected tissue different abundances were found for 49 proteins. The significantly upregulated proteins in the tissue that contained interstitial stroma (e.g. capillaries and fibroblasts) were related to coagulation and a downregulation in cytoskeletal regulation.

When zooming in on the ischemic region, a significant difference was found for 13 proteins between the border and core of the infarct. The core region showed a higher abundance for structural proteins like elastin and transgelin, and a lower abundance for mitochondrial fission 1 protein and keratin, type I cytoskeletal 13 proteins. Furthermore, pathway analysis was performed for the significantly altered proteins using the Reactome database through EnrichR. The enriched pathways in the infarct core region compared to the unaffected tissue showed that the top 10 pathways, based on ranking of the combined score, are related to coagulation, inflammatory responses and integrin signaling. On the other hand, downregulated proteins were related to energy metabolism.

Finally, the comparisons within the infarct tissue showed pathways involved in RHO GTPase activation and prostaglandin synthesis demonstrating ongoing changes in the architecture and inflammation.

TABLE 5
Cellular components from conductive slides before and after MSI,
from frozen and FFPE tissue, respectively. The top 10 most significant
components were clustered and shown in alphabetical order.

| | Frozen tissue | | | FFPE Tissue | | |
|---|---|---|---|---|---|---|
| | | After | | | After | |
| | | | Intelli | | | Intelli |
| | Before | ITO | Slide | Before | ITO | Slide |
| polarity | ITO | +/− | +/− | ITO | − | − |
| Cell junction | x | | | | x | x |
| Cytoplasm | x | x/x | x/x | x | | |
| Cytoplasmic | x | 0/x | | | | |
| vesicle | | | | | | |
| Cytoskeleton | | x/x | x/x | x | x | x |
| Mitochondrion | x | x/x | x/x | x | x | x |
| Protein-containing | | | | x | | |
| complex | | | | | | |
| Secretory granule | x | 0/x | 0/x | x | | |

### Example 3

The concept was repeated using mouse kidney tissue sections on an ITO coated glass slide, to verify that the principle can also be applied to lipids. Using the similar methodology it was possible to ablate material directly from the ITO slide and identify about 137 lipids using LC-MS.

### LITERATURE

1. Chughtai, K.; Heeren, R. M., Mass spectrometric imaging for 15 biomedical tissue analysis. Chem Rev 2010, 110 (5), 3237-77.
2. Datta, S.; Malhotra, L.; Dickerson, R.; Chaffee, S.; Sen, C. K.; Roy, S., Laser capture microdissection: Big data from small samples. Histol Histopathol 2015, 30 (11), 1255-69.
3. Banks, R. E.; Dunn, M. J.; Forbes, M. A.; Stanley, A.; Pappin, D.; Naven, T.; Gough, M.; Harnden, P.; Selby, P. J., The potential use of laser capture microdissection to selectively obtain distinct populations of cells for proteomics analysis—Preliminary findings. Electrophoresis 1999, 20 (4-5), 12.
4. Alberts, D.; Pottier, C.; Smargiasso, N.; Baiwir, D.; Maz-zucchelli, G.; Delvenne, P.; Kriegsmann, M.; Kazdal, D.; Warth, A.; De Pauw, E.; Longuespee, R., MALDI Imaging-Guided Microprote-omic Analyses of Heterogeneous Breast Tumors-A Pilot Study. Proteomics Clin Appl 2018, 12 (1).
5. Dilillo, M.; Pellegrini, D.; Ait-Belkacem, R.; de Graaf, E. L.; Caleo, M.; McDonnell, L. A., Mass Spectrometry Imaging, Laser Capture Microdissection, and LC-MS/MS of the Same Tissue Sec-tion. J Proteome Res 2017, 16 (8), 2993-3001.
6. Dewez, F.; Martin-Lorenzo, M.; Herfs, M.; Baiwir, D.; Mazzucchelli, G.; De Pauw, E.; Heeren, R. M. A.; Balluff, B., Precise co-registration of mass spectrometry imaging, histology, and laser microdissection-based omics. Anal Bioanal Chem 2019, 411 (22), 5647-5653.
7. Longuespee, R.; Alberts, D.; Pottier, C.; Smargiasso, N.; Mazzucchelli, G.; Baiwir, D.; Kriegsmann, M.; Herfs, M.; Kriegsmann, J.; Delvenne, P.; De Pauw, E., A laser microdissection-based workflow for FFPE tissue microproteomics: Important con-siderations for small sample processing. Methods 2016, 104, 154-62.
8. Ly, A.; Buck, A.; Balluff, B.; Sun, N.; Gorzolka, K.; Feuchtinger, A.; Janssen, K. P.; Kuppen, P. J.; van de Velde, C. J.; Weirich, G.; Erlmeier, F.; Langer, R.; Aubele, M.; Zitzelsberger, H.; McDonnell, L.; Aichler, M.; Walch, A., High-mass-resolution MALDI mass spectrometry imaging of metabolites from formalin-fixed paraffin-embedded tissue. Nat Protoc 2016, 11 (8), 1428-43.
9. Kuleshov, M. V.; Jones, M. R.; Rouillard, A. D.; Fernan-dez, N. F.; Duan, Q.; Wang, Z.; Koplev, S.; Jenkins, S. L.; Jagodnik, K. M.; Lachmann, A.; McDermott, M. G.; Monteiro, C. D.; Gunder-sen, G. W.; Ma′ayan, A., Enrichr: a comprehensive gene set enrichment analysis web server 2016 update. Nucleic Acids Res 2016, 44 (W1), W90-7.
10. Strohalm, M.; Hassman, M.; Kosata, B.; Kodicek, M., mMass data miner: an open source alternative for mass spectrometric data analysis. Rapid Commun Mass Spectrom 2008, 22 (6), 905-8.
11. Vogel, A.; Horneffer, V.; Lorenz, K.; Linz, N.; Hüttmann, G.; Gebert, A., Chapter 5: Principles of Laser Microdissection and Catapulting of Histologic Specimens and Live Cells. Methods in Cell Biology 2007, 82, 53.
12. Schutze, K.; Niyaz, Y.; Stich, M.; Buchstaller, A., Chapter 23: Noncontact Laser Microdissection and Catapulting for Pure Sample Capture. Methods in Cell Biology 2007, 82, 26.
13. Kuhn, D. E.; Roy, S.; Radtke, J.; Khanna, S.; Sen, C. K., Laser microdissection and capture of pure cardiomyocytes and fibroblasts from infarcted heart regions: perceived hyperoxia induces p21 in peri-infarct myocytes. Am J Physiol Heart Circ Physiol 2007, 292 (3), H1245-53.
14. Craven, R. A.; Totty, N.; Harnden, P.; Selby, P. J.; Banks, R. E., Laser capture microdissection and two-dimensional poly-acrylamide gel electrophoresis: evaluation of tissue preparation and sample limitations. Am J Pathol 2002, 160 (3), 815-22.
15. Vertommen, A.; Panis, B.; Swennen, R.; Carpentier, S. C., Evaluation of chloroform/methanol extraction to facilitate the study of membrane proteins of non-model plants. Planta 2010, 231 (5), 1113-25.
16. Piehowski, P. D.; Zhu, Y.; Bramer, L. M.; Stratton, K. G.; Zhao, R.; Orton, D. J.; Moore, R. J.; Yuan, J.; Mitchell, H. D.; Gao, Y.; Webb-Robertson, B. M.; Dey, S. K.; Kelly, R. T.; Burnum-Johnson, K. E., Automated mass spectrometry imaging of over 2000 proteins from tissue sections at 100-mum spatial resolution. Nat Commun 2020, 11 (1), 8.
17. Zhu, Y.; Dou, M.; Piehowski, P. D.; Liang, Y.; Wang, F.; Chu, R. K.; Chrisler, W. B.; Smith, J. N.; Schwarz, K. C.; Shen, Y.; Shukla, A. K.; Moore, R. J.; Smith, R. D.; Qian, W. J.; Kelly, R. T., Spatially Resolved Proteome Mapping of Laser Capture Microdis-sected Tissue with Automated Sample Transfer to Nanodroplets. Mol Cell Proteomics 2018, 17 (9), 1864-1874.
18. Quanico, J.; Franck, J.; Wisztorski, M.; Salzet, M.; Four-nier, I., Integrated mass spectrometry imaging and omics workflows on the same tissue section using grid-aided, parafilm-assisted microdissection. Biochim Biophys Acta Gen Subj 2017, 1861 (7), 1702-1714.
19. Stauber, J.; MacAleese, L.; Franck, J.; Claude, E.; Snel, M.; Kaletas, B. K.; Wiel, I. M.; Wisztorski, M.; Fournier, I.; Heeren, R. M., On-tissue protein identification and imaging by MALDI-ion mobility mass spectrometry. J Am Soc Mass Spectrom 2010, 21 (3), 338-47.
20. Groseclose, M. R.; Andersson, M.; Hardesty, W. M.; Cap-rioli, R. M., Identification of proteins directly from tissue: in situ tryptic digestions coupled with imaging mass spectrometry. J Mass Spectrom 2007, 42 (2), 254-62.
21. Griffiths, R. L.; Creese, A. J.; Race, A. M.; Bunch, J.; Cooper, H. J., LESA FAIMS Mass Spectrometry for the Spatial Profiling of Proteins from Tissue. Anal Chem 2016, 88 (13), 6758-66.
22. Towers, M. W.; Karancsi, T.; Jones, E. A.; Pringle, S. D.; Claude, E., Optimised Desorption Electrospray Ionisation Mass Spectrometry Imaging (DESI-MSI) for the Analysis of Proteins/Peptides Directly from Tissue Sections on a Travelling Wave Ion Mobility Q-ToF. J Am Soc Mass Spectrom 2018, 29 (12), 2456-2466.
23. Mezger, S. T. P., Mingels, A. M. A., Bekers, O., Heeren, R. M. A., and Cillero-Pastor, B. (2021). Mass Spectrometry Spatial-Omics on a Single Conductive Slide. Anal Chem 93(4), 2527-2533. doi: 10.1021/acs.analchem.0c04572.

1. A method for the detection of analytes in a tissue sample, comprising the steps of:
applying the tissue sample to a glass slide having an electrically conductive coating;
carrying out a mass spectrometry imaging (MSI) analysis of the tissue sample on the glass slide;
subjecting the tissue sample to laser capture microdissection (LCM) to dissect sample material from the tissue sample on the same glass slide; and
analysing the dissected sample material to detect the analytes,
wherein the laser capture microdissection is carried out in ablation mode.

2. The method according to claim 1, wherein the analytes are proteins, lipids, glycans or metabolites.

3. The method according to claim 1, wherein the analytes are proteins.

4. The method according to claim 3, wherein the proteins originate from cellular and sub-cellular components, preferably selected from mitochondria, cytoplasm, nuclei and cytoskeleton or they can be extracellular matrix proteins.

5. The method according to claim 1, wherein the glass slide is coated with an indium tin oxide coating.

6. The method according to claim 1, wherein the mass spectrometry imaging analysis is selected from MALDI (Matrix-Assisted Laser Desorption-Ionization) and SIMS (Secondary Ion Mass Spectrometry).

7. The method according to claim 6, wherein the MSI analysis is MALDI MSI, and wherein the method further comprises a step of applying a matrix onto the tissue sample before carrying out the MALDI-MSI analysis and a step of removing the matrix after carrying out the MALDI-MSI analysis.

8. The method according to claim 7, wherein the matrix is selected from α-cyano-4-hydroxycinnamic acid (CHCA), sinapic acid (4-hydroxy-3,5-dimethoxycinnamic acid), 2,5-dihydroxybenzoic acid (DHB), 2-(4-hydroxy phenyl azo) benzoic acid (HABA), succinic acid, 2,6-dihydroxy acetophenone, ferulic acid, caffeic acid (3,4-dihydroxy-cinnamic acid), 2,4,6-trihydroxy acetophenone, 3-hydroxypicolinic acid, 2-aminobenzoic acid, nicotinic acid, trans-3-indoleacrylic acid, isovanillin, dithranol, 9-aminoacridine (9-AA) and β-carboline (Norharmane).

9. The method according to claim 1, wherein the MSI analysis is used to define a region of interest (ROI).

10. The method of claim 9, wherein the ROI is ablated in the LCM step.

11. The method of claim 1, wherein the ablated tissue sample is collected.

12. The method of claim 11 wherein the collected tissue sample is treated for storage or further analysis.
